(19) United States
(12) Patent Application Publication
(10) Pub. No.: US 2019/0343483 A1
(43) Pub. Date: Nov. 14, 2019

(54) SYSTEMS, METHODS, AND APPARATUSES FOR FLUID INGRESS DETECTION FOR ULTRASOUND TRANSDUCERS

(71) Applicant: KONINKLIJKE PHILIPS N.V., EINDHOVEN (NL)

(72) Inventors: Sujith Kanakasabhapathi, Acton, MA (US); Bernard Joseph Savord, Andover, MA (US); Hoi-Cheong Steve Sun, Lexington, MA (US); Nikolas Keith Ledoux, Merrimack, NH (US); Barry Carl Scheirer, McAlisterville, PA (US); William John Ossmann, Acton, MA (US); Satish Sanjay Singh, Everett, MA (US); Livia Rodriguez, Cary, NC (US)

(21) Appl. No.: 16/474,169

(22) PCT Filed: Dec. 28, 2017
(86) PCT No.: PCT/EP2017/084762

§ 371 Date: Jun. 27, 2019

Related U.S. Application Data
(60) Provisional application No. 62/442,064, filed on Jan. 4, 2017.

(51) Int. Cl. A61B 8/00; G01S 7/52

(52) U.S. Cl. CPC A61B 8/4444; A61B 8/58; G01N 27/048; G01S 7/52079; G01S 7/5205

(57) ABSTRACT

Systems, methods, and apparatuses for detecting fluid ingress in ultrasound probes are disclosed. A fluid ingress may be detected by a change in resistance, capacitance, and/or current of a fluid ingress detector of a fluid ingress detection assembly. A fluid ingress detector that includes a soluble material is disclosed. A fluid ingress detector that includes a galvanic sensor is disclosed. The fluid ingress detector may provide a signal indicating a fluid ingress to an ultrasound imaging system and/or a maintenance diagnostic system.

### BACKGROUND

One cause of ultrasound probe failure is fluid ingress. That is, water and/or other fluids leak into the interior of the probe. The fluids may cause electrical shorts that may damage components of the ultrasound probe and/or corrode internal components of the probe. Although external surfaces of ultrasound probes may be exposed to fluids during imaging exams, the fluid barriers of ultrasound probes are often compromised during cleaning rather than during exams. Ultrasound probes may need to be disinfected between exams to prevent transmission of diseases between patients. Probes may be disinfected by enzymatic cleaning and/or high level disinfection. Disinfection procedures may include the use of fluids including strong acids, bases, and/or corrosive compounds (e.g., alcohol, hydrogen peroxide, ammonia, bleach). During disinfection, connectors and/or other components of the ultrasound probe not designed to resist fluid ingress may be exposed to water, disinfectants, and/or other fluids. Users may also bend ultrasound components (e.g., neck and sheath of a transesophageal echo probe, coaxial cable) beyond their designed range of motion during cleaning. This may compromise fluid ingress barriers of the ultrasound probe.

Currently, it is often impossible for a user or field technician to detect fluid ingress without disassembling at least a portion of the ultrasound probe. This may make it difficult to diagnose a maintenance issue when an ultrasound probe is malfunctioning and delay repairs. If fluid ingress is left uncorrected, the ultrasound probe may be damaged beyond repair and need to be fully replaced. Full replacement of an ultrasound probe may increase costs and/or downtime of an ultrasound imaging system.

### SUMMARY

According to an exemplary embodiment of the disclosure, an ultrasound probe may include a transducer assembly including an array of transducer elements, a housing enclosing at least a portion of the transducer assembly, a cable coupled to the housing, a connector coupled to the cable, the connector configured to couple the ultrasound probe to an ultrasound imaging system, and a fluid ingress detection assembly that may include a sense circuit, and a fluid ingress detector that may be configured to change from a first state to a second state responsive to ingress of fluid in an interior of the ultrasound probe, and the sense circuit may be operable to detect the change from the first state to the second state and generate a signal indicative of fluid ingress.

In one exemplary embodiment, the fluid ingress detector may include a first conductive line, a second conductive line, wherein the first and second conductive lines may be electrically coupled to the sense circuit, a conductive cantilever electrically coupled to the first conductive line, and a soluble epoxy, wherein the soluble epoxy may electrically couple the conductive cantilever to the second conductive line. In some examples, the fluid ingress detector may further include a spring coupled between the first conductive line and the conductive cantilever, wherein the spring may bias the conductive cantilever away from the second conductive line. In some examples, the fluid ingress detector may further include a lever coupled between the first conductive line and the conductive cantilever, wherein the lever may bias the conductive cantilever away from the second conductive line.

In an alternative exemplary embodiment, the soluble epoxy may electrically isolate the conductive cantilever and the second conductive line of the fluid ingress detector. In some examples, the fluid ingress detector may further comprise a spring coupled between the first conductive line and the conductive cantilever, wherein the spring may bias the conductive cantilever toward the second conductive line. In some examples, the fluid ingress detector may further comprise a lever coupled between the first conductive line and the conductive cantilever, wherein the lever may bias the conductive cantilever toward the second conductive line. In some examples, the conductive cantilever is molded such that the conductive cantilever may be biased toward the second conductive line.

According to another exemplary embodiment of the disclosure, the fluid ingress detector may include a light source, a soluble epoxy coating the light source, wherein the soluble epoxy may block a light of the light source, and a photodetector electrically coupled to the sense circuit, wherein the photodetector may be configured to detect the light from the light source when at least some of the soluble epoxy dissolves and may generate at least one of a voltage and a current responsive to detecting the light of the light source.

According to a further exemplary embodiment of the disclosure, the fluid ingress detector may include a first portion comprising a first material and a second portion comprising a second material different from the first material, wherein the first portion and second portion may comprise a galvanic sensor. The first and second portions may form a closed circuit when a fluid contacts the first and second portions and the sense circuit may be configured to sense at least one of a voltage and a current from the galvanic sensor when the closed circuit is formed.

According to a further exemplary embodiment of the disclosure, an ultrasound imaging system may include an ultrasound probe including a capacitor including a soluble dielectric and a dielectric property sensing circuit electrically coupled to the ultrasound probe, wherein the dielectric property sensing circuit may be configured to detect a change in a dielectric property of the capacitor when at least some of the soluble dielectric reacts.

### BRIEF DESCRIPTION OF THE DRAWINGS

FIG. 1 is a block diagram of an ultrasound imaging system according to an embodiment of the disclosure.

FIG. 2 is a schematic illustration of an exploded view of an ultrasound probe according to an embodiment of the disclosure.

FIGS. 3A and 3B illustrate an example of a fluid ingress detector according to an embodiment of the disclosure.

FIGS. 4A and 4B illustrate an example of a fluid ingress detector according to another embodiment of the disclosure.

FIGS. 5A and 5B illustrate examples of fluid ingress detectors according to embodiments of the disclosure.

FIG. 6A is a functional block diagram of a system including a fluid ingress detector according to an embodiment of the disclosure.

FIG. 6B is a functional block diagram of a system for detecting a fluid ingress according to an embodiment of the disclosure.

FIG. 7 is an illustration of an example capacitive structure that may be used to implement capacitors shown in FIGS. 6A and 6B according to embodiments of the disclosure.

FIGS. 8A and 8B are illustrations of cross-sectional views of the capacitive structure shown in FIG. 7 according to embodiments of the disclosure.

FIG. 9 is an illustration of an example fluid ingress detector according to an embodiment of the disclosure.

FIG. 10 shows a circuit diagram of an exemplary sense circuit according to embodiments of the disclosure.

### DETAILED DESCRIPTION

The following description of certain exemplary embodiments is merely exemplary in nature and is in no way intended to limit the invention or its applications or uses. In the following detailed description of embodiments of the present systems and methods, reference is made to the accompanying drawings which form a part hereof, and in which are shown by way of illustration specific embodiments in which the described systems and methods may be practiced. These embodiments are described in sufficient detail to enable those skilled in the art to practice the presently disclosed systems and methods, and it is to be understood that other embodiments may be utilized and that structural and logical changes may be made without departing from the spirit and scope of the present system.

The following detailed description is therefore not to be taken in a limiting sense, and the scope of the present system is defined only by the appended claims. The leading digit(s) of the reference numbers in the figures herein typically correspond to the figure number, with the exception that identical components which appear in multiple figures are identified by the same reference numbers. Moreover, for the purpose of clarity, detailed descriptions of certain features will not be discussed when they would be apparent to those with skill in the art so as not to obscure the description of the present system.

According to an embodiment of the disclosure, an ultrasound probe may include one or more fluid ingress detectors. The ultrasound probe may include a housing, sheath, cable, connector and/or coupler. The fluid ingress detector may be located in the interior of the ultrasound probe. For example, a fluid ingress detector may be located in an interior of the housing at or near where the cable couples to the housing. In another example, a fluid ingress detector may be located in an interior of the connector that couples the ultrasound probe to an ultrasound imaging system. The detector may provide maintenance diagnostic information to a user and/or a field engineer. In some embodiments, the detector may provide maintenance diagnostic information via a user interface and/or display included in an ultrasound imaging system coupled to the ultrasound probe. In some embodiments, the detector may trigger a visual or audio indicator included in the ultrasound probe to provide maintenance diagnostic information. In some embodiments, the detector may provide maintenance diagnostic information to a maintenance diagnostic system coupled to the ultrasound probe. The maintenance diagnostic information may allow appropriate repairs to the ultrasound probe to be completed. The maintenance diagnostic information may allow the field engineer to detect probe misuse by the user and educate the user on proper disinfecting and/or cleaning procedures that are less likely to cause fluid ingress.

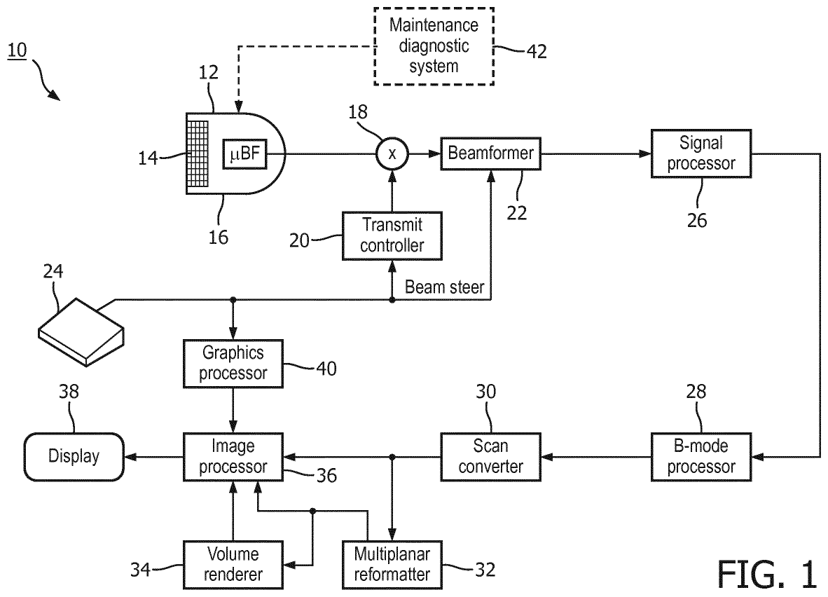

Referring to FIG. 1, an ultrasound imaging system 10 constructed in accordance with the principles of the present invention is shown in block diagram form. In the ultrasonic diagnostic imaging system of FIG. 1, an ultrasound probe 12 includes a transducer array 14 for transmitting ultrasonic waves and receiving echo information. A variety of transducer arrays are well known in the art, e.g., linear arrays, convex arrays or phased arrays. The transducer array 14, for example, can include a two dimensional array (as shown) of transducer elements capable of scanning in both elevation and azimuth dimensions for 2D and/or 3D imaging. The transducer array 14 is coupled to a microbeamformer 16 in the probe 12 which controls transmission and reception of signals by the transducer elements in the array. In this example, the microbeamformer is coupled by the probe cable to a transmit/receive (T/R) switch 18, which switches between transmission and reception and protects the main beamformer 22 from high energy transmit signals. In some embodiments, the T/R switch 18 and other elements in the system can be included in the transducer probe rather than in a separate ultrasound system base. The transmission of ultrasonic beams from the transducer array 14 under control of the microbeamformer 16 is directed by the transmit controller 20 coupled to the T/R switch 18 and the beamformer 22, which receives input from the user's operation of the user interface or control panel 24. One of the functions controlled by the transmit controller 20 is the direction in which beams are steered. Beams may be steered straight ahead from (orthogonal to) the transducer array, or at different angles for a wider field of view. The partially beamformed signals produced by the microbeamformer 16 are coupled to a main beamformer 22 where partially beamformed signals from individual patches of transducer elements are combined into a fully beamformed signal.

The beamformed signals are coupled to a signal processor 26. The signal processor 26 can process the received echo signals in various ways, such as bandpass filtering, decimation, I and Q component separation, and harmonic signal separation. The signal processor 26 may also perform additional signal enhancement such as speckle reduction, signal compounding, and noise elimination. The processed signals are coupled to a B mode processor 28, which can employ amplitude detection for the imaging of structures in the body. The signals produced by the B mode processor are coupled to a scan converter 30 and a multiplanar reformatter 32. The scan converter 30 arranges the echo signals in the spatial relationship from which they were received in a desired image format. For instance, the scan converter 30 may arrange the echo signal into a two dimensional (2D) sector-shaped format, or a pyramidal three dimensional (3D) image. The multiplanar reformatter 32 can convert echoes which are received from points in a common plane in a volumetric region of the body into an ultrasonic image of that plane, as described in U.S. Pat. No. 6,443,896 (Detmer). A volume renderer 34 converts the echo signals of a 3D data set into a projected 3D image as viewed from a given reference point, e.g., as described in U.S. Pat. No. 6,530,885 (Entrekin et al.) The 2D or 3D images are coupled from the scan converter 30, multiplanar reformatter 32, and volume renderer 34 to an image processor 36 for further enhancement, buffering and temporary storage for display on an image display 38. The graphics processor 36 can generate graphic overlays for display with the ultrasound images. These graphic overlays can contain, e.g., standard identifying information such as patient name, date and time of the image, imaging parameters, and the like. For these purposes the graphics processor receives input from the user interface 24, such as a typed patient name. The user interface can also be coupled to the multiplanar reformatter 32 for selection and control of a display of multiple multiplanar reformatted (MPR) images.

In some embodiments, the user interface 24 may allow a user and/or a field technician to run maintenance diagnostics on one or more components of the ultrasound imaging system 10. For example, a field technician may be able to run test functions on the ultrasound probe 12 via the user interface 24. The ultrasound system 10 may provide error codes and/or other maintenance diagnostic information to the filed technician via the display 38. In some embodiments, the user interface 24 and/or ultrasound probe 12 may provide maintenance diagnostic indicators (e.g., LCD screen, LED, audio signal). In some embodiments, the field technician may couple one or more components of the ultrasound imaging system 10 to a separate maintenance diagnostic system 42 to acquire maintenance diagnostic information on the component (e.g., ultrasound probe 12).

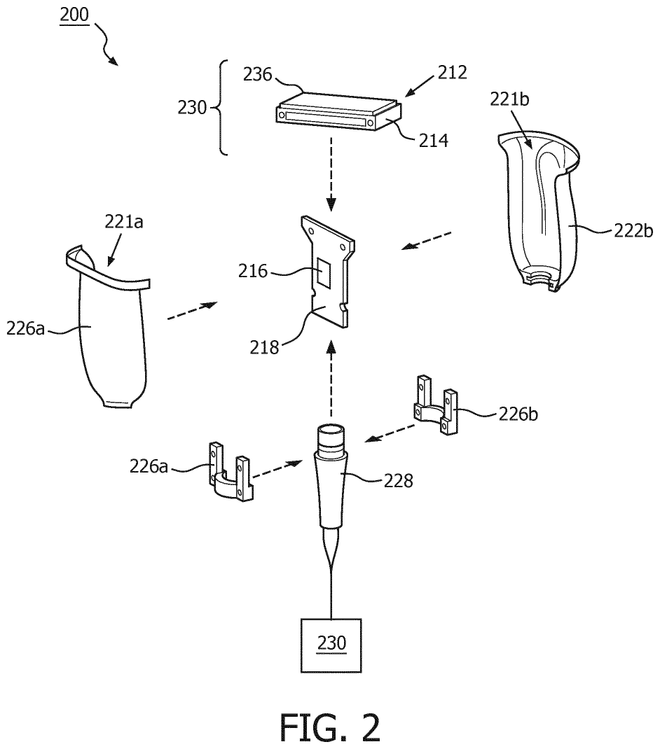

FIG. 2 is a schematic illustration of an exploded view of an ultrasound probe 200 that may be used to implement ultrasound probe 12 shown in FIG. 1, according to an embodiment of the disclosure. As used herein, distal refers to an end of the ultrasound probe that is typically closest to and/or in contact with a subject or object to be imaged during use. Proximal refers to an end of the ultrasound probe that is typically farther from the subject or object to be imaged and/or closer to an ultrasound imaging system (not shown) during use. The ultrasound probe 200 may include a housing 222 which may form the handle portion of the probe that is held by a sonographer during use. The housing 222 is shown as having two portions 222a-b, which may be configured to mate to form the housing 222. However, the housing 222 may be a unitary body and/or be composed of more than two portions configured to mate in some embodiments. When the two portions 222a-b of the housing 222 are joined, the housing 222 may define an opening (not numbered) at a distal end of the probe 200 that may expose at least a portion of a lens 236 of a transducer assembly 230. The transducer assembly 230 may include the lens 236 at the distal end, a transducer stack 212 on the proximal side of the lens 236, and a backing subassembly 214 on the proximal side of the transducer stack 212. The transducer stack 212 may be between the lens 236 and the backing subassembly 214. The transducer stack 212 may include an array of transducer elements, for example a 1D or 2D array of piezoelectric, CMUT or another type of transducer elements configured to transmit ultrasonic waves and receive ultrasound echoes. The transducer assembly 230 may include a flexible circuit and/or other electrical components (not shown). In some embodiments, the flexible circuit may be included in the transducer stack 212. The flexible circuit and/or electrical components may couple the transducer or other components of the transducer stack 212 to other electrical components of the ultrasound probe 210. The backing subassembly 214 may attenuate acoustic reverberations from the back of the transducer stack 212 and/or may conduct heat developed in the transducer stack 212 away from the distal end of the probe 200. The backing subassembly 214 may include a graphite block in some embodiments. Although the transducer assembly 230 is shown as having a substantially rectangular shape, the transducer assembly 230 may have other shapes. Example suitable shapes include, but are not limited to, a dome, an arc, and a half-cylinder. The shape of the transducer assembly 230 may be determined, at least in part, by the ultrasound imaging application (e.g., thoracic, cardiac, esophageal).

The probe 200 may include a PCA 218 may include electrical circuits and/or other electrical components for operation of the ultrasound probe 200 (e.g., for applying voltage to the transducer elements for generating ultrasonic waves). In some examples, the PCA 218 may be coupled to the transducer assembly 230. The PCA 218 may be coupled to a flexible circuit (not shown) or other electrical components of the transducer assembly 230. The PCA 218 may be coupled to the housing 222. In some embodiments, the probe 200 may include two or more PCAs 218.

In some embodiments, the PCA 218 may include a fluid ingress detection assembly 216. The fluid ingress detection assembly 216 may include springs, levers, soluble capacitors, soluble epoxy, light emitting diodes, light sensors, galvanic sensors, a battery, a sense circuit, and/or other elements (e.g., wires, traces, latches, clock circuit). In some embodiments, one or more components of the fluid ingress detection assembly 216 may be coupled to the PCA 218, and in some embodiments between the PCA 218 and the interior surface 221 of the housing 222. In some embodiments, components of the fluid ingress detection assembly 216 may be located on another internal component (e.g., transducer stack 212, backing subassembly 214, housing 222) and electrically coupled to the PCA 218 and/or a flexible circuit of the transducer stack 212. In some embodiments, the probe 200 may include multiple fluid ingress detection assemblies 216. The fluid ingress detection assembly 216 may be positioned near locations vulnerable to fluid ingress (e.g., seam in the housing) and/or internal components sensitive to fluid ingress (e.g., electrical circuitry).

At the proximal end of the probe 200 and extending therefrom may be a cable 228 of the ultrasound probe 200. In some embodiments, the cable 228 may be a coaxial cable. In some embodiments, the cable 228 may be clamped to a proximal end of the PCA 218 by a clamp 226a-b. Other attachment methods may also be used. The cable 228 may include a connector 230 at a proximal end. The connector 230 may couple the probe 200 to an ultrasound imaging system such as ultrasound imaging system 10 shown in FIG. 1.

In some embodiments, the fluid ingress detection assembly 216 or portions of the fluid ingress detection assembly 216 may be included in the cable 228 and/or connector 230. For example, one or more fluid ingress detectors may be included in the connector 230 and electrically coupled via the cable 228 to another component of the fluid ingress detection assembly 216 included with the PCA 218. In another example, the fluid ingress detection assembly 216 may be included in the connector 230 and may communicate with an ultrasound imaging system via the connector 230 when the ultrasound probe 200 is coupled to the ultrasound imaging system.

The PCA 218, at least a portion of the transducer assembly 230, and/or other internal components of the probe 200 may be enclosed in the housing 222. The housing 222 may include two separate portions 222a-b that may be configured to fit together with each other to form an impervious housing to protect the ultrasound components from electromagnetic field interference, liquids, and/or debris. The housing 222 may comprise plastic, metal, rubber, and/or a combination of materials. In some embodiments, the housing 222 may be configured to enclose the transducer stack 212 and backing subassembly 214 of the transducer assembly 230 while leaving at least a portion of the lens 236 exposed.

Although ultrasound probe 200 as shown in FIG. 2 is configured as a hand-held probe, ultrasound probe 200 may be configured as an intravaginal probe, a transesophageal echo probe, and/or other ultrasound probe type. Ultrasound probe 200 may include all or some of the same components shown in FIG. 2, but the shape and/or dimensions of one or more components may be altered based on the desired application. For example, an intravaginal probe may have a dome-shaped lens 236 and/or transducer stack 212. In another example, a transesophageal probe may have an elongated, flexible housing 222.

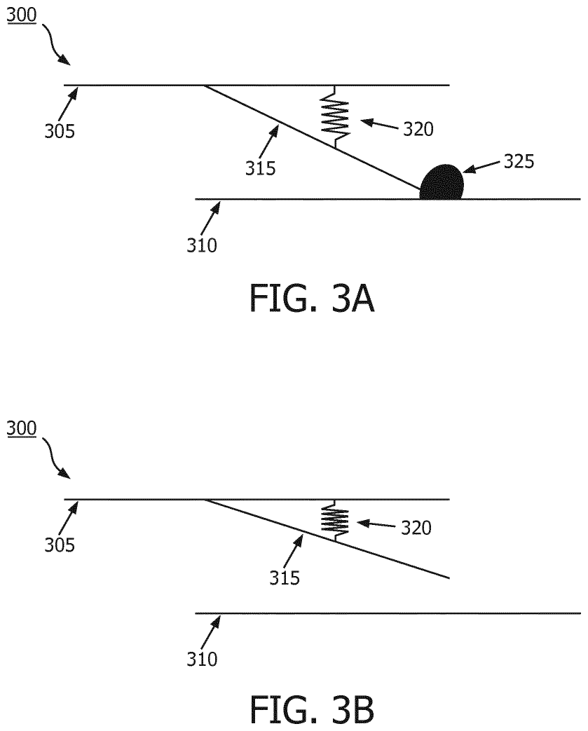

FIGS. 3A and 3B illustrate components of an example of a fluid ingress detection assembly according to an embodiment of the disclosure. The fluid ingress detection assembly of the example in FIGS. 3A and 3B includes a fluid ingress detector 300. Fluid ingress detector 300 may include a first conductive line 305, a conductive cantilever 315 electrically coupled to the first conductive line 305, and a second conductive line 310. The conductive cantilever 315 may be electrically coupled to the second conductive line 310 by a soluble epoxy 325. In some embodiments, the soluble epoxy 325 electrically couples the conductive cantilever 315 to the second conductive line 310 by maintaining the conductive cantilever 315 in physical contact with the second conductive line 310. In some embodiments, the soluble epoxy 325 or a portion thereof may be disposed between the conductive cantilever 315 and the second conductive line 310. In such embodiments, the soluble epoxy 325 may be electrically conductive and may thus electrically couple the conductive cantilever 315 to the second conductive line 310 even if the conductive cantilever 315 is not physically in contact with the second conductive line 310. The conductive cantilever 315 may be mechanically biased away from the second conductive line. For example, the fluid ingress detector 300 may include a spring 320 coupled between the first conductive line 305 and the conductive cantilever 315, which biases the conductive cantilever 315 away from the second conductive line 310. The soluble epoxy 325 acts against the mechanical bias to maintain the conductive cantilever 315 in electrical contact with the second conductive line.

As illustrated in FIG. 3A, prior to a fluid ingress, the conductive cantilever 315 may close a circuit that includes the first and second conductive lines 305, 310 by virtue of the electrical coupling provided by the soluble epoxy 325. The spring 320 may be in a strained state again by virtue of the soluble epoxy 325 which acts against the mechanical bias applied by the spring 320. FIG. 3B illustrates the fluid ingress detector 300 after a fluid ingress. At least some of the soluble epoxy 325 has been dissolved, and the spring 320 is in a resting or partially unstrained state. In this resting or partially unstrained state, in the absence of the soluble epoxy 325 acting against the spring, the biasing force of the spring acts freely on the conductive cantilever 315 to bias the conductive cantilever 315 away from the second conductive line 310 and to physically separate the conductive cantilever 315 from the second conductive line 310. Thus, as shown in FIG. 3B, the conductive cantilever 315 is no longer electrically coupled to the second conductive line 310. Thus, the circuit including the conductive cantilever 315 and the first and second conductive lines 305, 310 is open. While the spring 320 in this example is shown between the first conductive line 305 and the conductive cantilever 315, in other examples, the spring may be provided between the conductive cantilever 315 and the second conductive line 310. A sense circuit (not shown), may be coupled to the fluid ingress detector 300. The sense circuit may detect a change in voltage, resistance, and/or current between the closed circuit shown in FIG. 3A and the open circuit shown in FIG. 3B. The detected change in voltage, resistance, and/or current may cause the sense circuit to transmit a signal which produces an indication of fluid ingress. The signal may be provided to another component within the ultrasound probe which is operable to provide the indication (e.g., in the form of illuminated light connected to the housing, an audible indication provided by a speaker, or a tactile indication provided by a vibrator built into the probe) and/or an external system coupled to the ultrasound probe (e.g., in the form of a visual indication provided on a display of an ultrasound scanner).

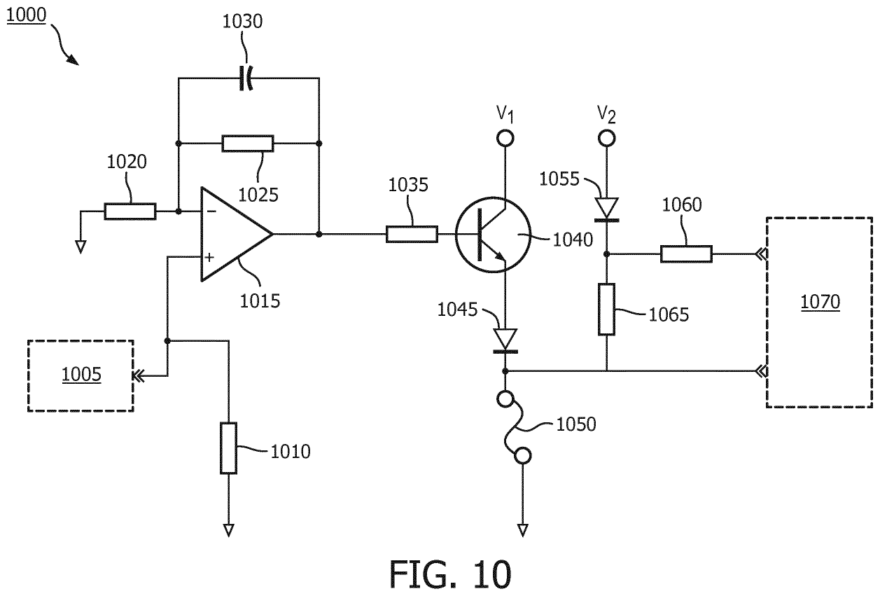

An example sense circuit is shown in FIG. 10 and is described in more detail below. However, it will be understood to those skilled in the art that a variety of sense circuits may be suitable for implementing a sense circuit coupled to the fluid ingress detector 300. For example, a current sensing circuit or a voltage comparator circuit may be used as a sense circuit.

The first and second conductive lines 305, 310 may be implemented as wires and/or plates in some embodiments. The first and second conductive lines 305, 310 may be included on a PCA in some embodiments. In some embodiments, the first and second conductive lines 305, 310 may be included on separate PCA's. In some embodiments, the first and/or second conductive lines 305, 310 are separate from a PCA and are electrically coupled to one or more PCA's.

The conductive cantilever 315 may be implemented as a wire or a strip of conductive material in some embodiments. In some embodiments, the conductive cantilever 315 is shaped, e.g., bent and/or molded into a shape which has a natural bias away from the second conductive line 310. For example, the conductive cantilever 315 may be shaped into a pre-loaded state in which the conductive cantilever 315 tends towards a shape that would separate the first and second conductive lines in the absence of the holding force applied by the soluble epoxy. In some embodiments, the conductive cantilever 315 is biased away from the second conductive line 310 by the spring 320 or another biasing mechanism now known or later developed. In some embodiments, the conductive cantilever 315 and the first and second conductive lines 305, 310 include copper or a copper-beryllium alloy. However, other conductive materials may be used (e.g., conductive polymers, gold).

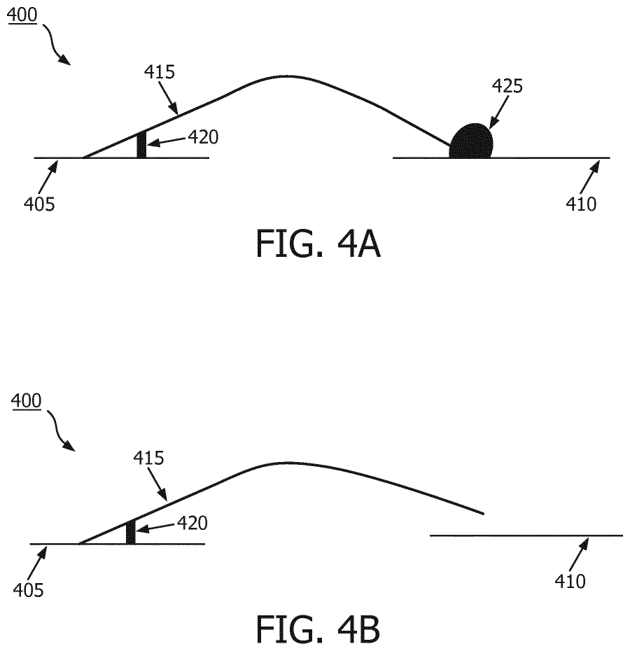

FIGS. 4A and 4B illustrate another example of a fluid ingress detector 400 according to an embodiment of the disclosure. Fluid ingress detector 400 may be used to implement a fluid ingress detector of the fluid ingress detection assembly 216 shown in FIG. 2 in some embodiments. Fluid ingress detector 400 may include a first conductive line 405, a conductive cantilever 415 electrically coupled to the first conductive line 405, and a second conductive line 410. The conductive cantilever 415 may be electrically coupled to the second conductive line 410 by a soluble epoxy 425. The fluid ingress detector 400 may further include a biasing mechanism in the form of a post 420 coupled between the first conductive line 405 and the conductive cantilever 415. In some embodiments, the post 420 may be compressible and may elongate in the absence of the holding force applied by the soluble epoxy 425. In some embodiments, the post 420 is rigid and acts as a fulcrum of the cantilever 415, which may be a flexible member naturally biased away from the second conductive line 405 but held in physical and/or electrical contact by the soluble epoxy 425.

As illustrated in FIG. 4A, prior to a fluid ingress, the conductive cantilever 415 may close a circuit that includes the first and second conductive lines 405, 410. FIG. 4B illustrates the fluid ingress detector 400 after a fluid ingress. At least some of the soluble epoxy 425 has been dissolved. The conductive cantilever 415 is no longer electrically coupled to the second conductive line 410. Thus, the circuit including the conductive cantilever 415 and the first and second conductive lines 405, 410 is open.

A sense circuit (not shown), may be coupled to the fluid ingress detector 400. The sense circuit may detect a change in voltage, resistance, and/or current between the closed circuit shown in FIG. 4A and the open circuit shown in FIG. 4B. The detected change in voltage, resistance, and/or current may cause the sense circuit to transmit a signal indicating a fluid ingress. The signal may be provided to another component within the ultrasound probe and/or an external system coupled to the ultrasound probe.

The first and second conductive lines 405, 410 may be implemented as wires and/or plates in some embodiments. The first and second conductive lines 405, 410 may be included on a PCA in some embodiments. In some embodiments, the first and second conductive lines 405, 410 may be included on separate PCA's. In some embodiments, the first and/or second conductive lines 405, 410 are separate from a PCA and are electrically coupled to one or more PCA's.

The conductive cantilever 415 may be implemented as a wire or a strip of conductive material in some embodiments. In some embodiments, the conductive cantilever 415 is bent and/or molded to have a bias away from the second conductive line 410. In some embodiments, the conductive cantilever 415 is biased away from the second conductive line such that the post 420 is optional. For example, the conductive cantilever 415 may be shaped into a pre-loaded state in which the conductive cantilever 415 tends towards a shape that would separate the first and second conductive lines in the absence of the holding force applied by the soluble epoxy. In some embodiments, the conductive cantilever 415 and the first and second conductive lines 405, 410 include copper or a copper-beryllium alloy. However, other conductive materials may be used (e.g., conductive polymers, gold).

Although the fluid ingress detectors 300 and 400 are described as closing a circuit in response to a fluid ingress, in some embodiments, fluid ingress detectors 300 and 400 may be configured to open a circuit in response to a fluid ingress. For example, soluble epoxy 325 of fluid ingress detector 300 may electrically isolate conductive cantilever 315 and conductive line 310. Soluble epoxy 325 may secure conductive cantilever 315 such that spring 320 is in a compressed state. After at least a portion of soluble epoxy 325 dissolves, the spring 320 may urge conductive cantilever 315 into electrical contact with conductive line 310. In another example, conductive cantilever 415 of fluid ingress detector 400 may be molded to be biased toward conductive line 410 and/or conductive cantilever 415 may be biased toward conductive line 410 by lever 420. The soluble epoxy 425 may electrically isolate conductive line 410 and conductive cantilever 415. After at least some of the soluble epoxy 425 dissolves due to a fluid ingress, conductive cantilever 415 may come into electrical contact with conductive line 410.

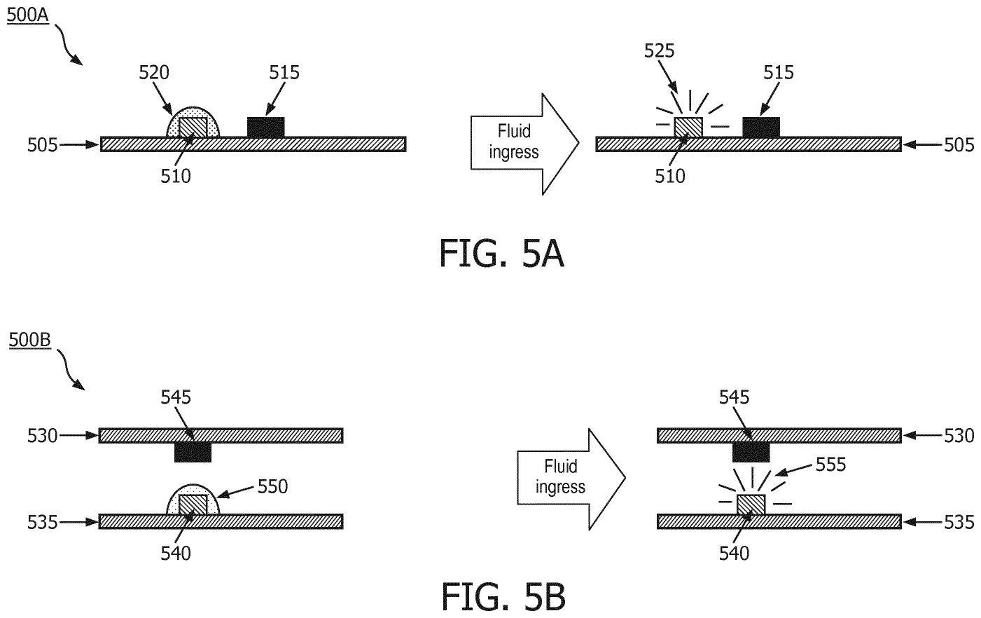

FIGS. 5A and 5B illustrate examples of fluid ingress detectors 500A and 500B according to embodiments of the disclosure. Fluid ingress detectors 500A and 500B may be used to implement a fluid ingress detector for the fluid ingress detection assembly 216 shown in FIG. 2 in some embodiments. Fluid ingress detector 500A may be included with a PCA 505. The fluid ingress detector 500A may include a light source 510 (e.g., LED, quantum dot) and a photodetector 515 (e.g., charged-coupled device, photodiode). As shown on the left-hand side of FIG. 5A, the light source 510 may be coated in a soluble epoxy 520. After fluid ingress, at least some of the soluble epoxy 520 may be dissolved, and light 525 emitting from the light source 510 may be detected by the photodetector 515 as shown on the right-hand side of FIG. 5A.

A sense circuit (e.g., the sense circuit shown in FIG. 10), may be coupled to the fluid ingress detector 500A. The sense circuit may detect a voltage and/or current from the photodetector 515. The detected voltage and/or current may cause the sense circuit to transmit a signal indicating a fluid ingress. The signal may be provided to another component within the ultrasound probe and/or an external system coupled to the ultrasound probe.

In some embodiments, the light source 510 may be a visible light source. In some embodiments, the light source 510 may be an infrared light source. The wavelength range of the light source 510 may be selected based, at least in part, on the properties of the soluble epoxy 520. For example, many epoxies strongly absorb in the infrared region. Selecting an infrared light source may allow a thinner coating of soluble epoxy 520 to be used over the light source 510 to block light 525 from reaching the photodetector 515 prior to a fluid ingress.

FIG. 500B is an illustration of a fluid ingress detector 500B. Fluid ingress detector 500B may include a light source 540 coated in a soluble epoxy 550 and a photodetector 545. In the arrangement shown in FIG. 5B, the light source 540 is included with PCA 535 and the photodetector 545 is included with PCA 530. Fluid ingress detector 500B may operate in a similar manner as fluid ingress detector 500A. As shown on the left-hand side of FIG. 5B, the light source 540 may be coated in a soluble epoxy 550. After fluid ingress, at least some of the soluble epoxy 550 may be dissolved, and light 555 emitting from the light source 540 may be detected by the photodetector 545 as shown on the right-hand side of FIG. 5B.

FIGS. 5A and 5B are two exemplary arrangements of a fluid ingress detector including light source and photodetector components. In some embodiments, a fluid ingress detector may include multiple light sources included on one or more PCA's. The light sources may be evenly spaced throughout an ultrasound probe and/or placed near areas vulnerable to fluid ingress. In some embodiments, a fluid ingress detector may include multiple photodetectors included on one or more PCA's. In some embodiments, there may be a greater number of light sources than photodetectors. In some embodiments, there may be a greater number of photodetectors than light sources.

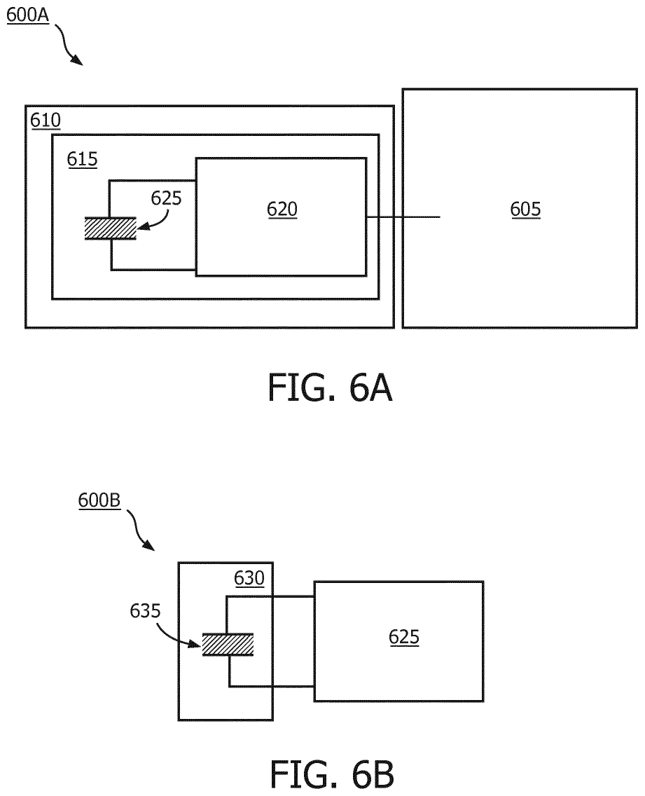

FIG. 6A is a functional block diagram of a system 600A including a fluid ingress detector 615 according to an embodiment of the disclosure. System 600A may include an ultrasound imaging system 605 coupled to an ultrasound probe 610. System 600A may be implemented with ultrasound imaging system 10 shown in FIG. 1 in some embodiments. In some embodiments, ultrasound probe 610 may be used to implement ultrasound probe 12 shown in FIG. 1. Ultrasound probe 610 may include fluid ingress detector 615. Fluid ingress detector 615 may include a dielectric property sensing circuit 620 coupled to a capacitor 625 including a soluble dielectric. In some embodiments, fluid ingress detector 615 may include multiple capacitors 625. At least some of the dielectric may react (e.g., dissolve, absorb fluid) if ultrasound probe 610 experiences a fluid ingress. The dielectric properties (e.g., capacitance, impedance) of capacitor 625 may change when some or all of the dielectric reacts. The change in dielectric properties may be detected by the dielectric property sensing circuit 620. Based on the detected change, the dielectric property sensing circuit 620 may transmit a signal indicating a fluid ingress. The signal may be provided to another component within the ultrasound probe 610 and/or the ultrasound system 605. In some embodiments, the ultrasound system 605 may be replaced by a maintenance diagnostic system such as maintenance diagnostic system 42 shown in FIG. 1. In some embodiments, ultrasound probe 610 may include multiple fluid ingress detectors 615.

FIG. 6B is a functional block diagram of a system 600B for detecting a fluid ingress according to an embodiment of the disclosure. System 600B may include an ultrasound probe 630. In some embodiments, ultrasound probe 630 may be used to implement ultrasound probe 12 shown in FIG. 1. Ultrasound probe 630 may include a capacitor 635 including a soluble dielectric. In some embodiments, ultrasound probe 630 may include multiple capacitors 635. At least some of the dielectric may react (e.g., dissolve, absorb fluid) if ultrasound probe 630 experiences a fluid ingress. The dielectric properties (e.g., capacitance, impedance) of capacitor 635 may change when some or all of the dielectric reacts and/or dissolves. The change in dielectric properties may be detected by an external dielectric property sensing system 625 coupled to ultrasound probe 630. In some embodiments, the external dielectric property sensing system 625 may be included with a maintenance diagnostic system, such as maintenance diagnostic device 42 shown in FIG. 1. In some embodiments, the external dielectric property sensing system 625 may be included with an ultrasound imaging system, such as ultrasound imaging system 10 shown in FIG. 1. Based on the detected change in dielectric properties, the dielectric property sensing device 625 may transmit a signal indicating a fluid ingress. The signal may be used to provide maintenance diagnostic information to a user or field engineer (e.g., error message, LED, audio signal).

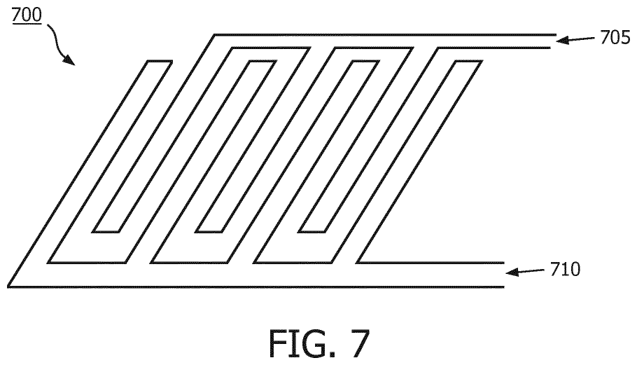

FIG. 7 is an illustration of an example capacitive structure 700 that may be used to implement capacitors 625, 635 according to embodiments of the disclosure. Capacitive structure 700 may include alternating PCA trace “fingers” which form two plates 705, 710 of the capacitive structure 700. Plate 705 and 710 may be spaced apart from one another. Although shown as straight lines, plates 705, 710 may be implemented as other shapes. For example, plates 705, 710 may be implemented as spirals or curved lines around other components included with the PCA. In some embodiments, a PCA may include multiple capacitive structures 700. In some embodiments, the capacitive structure 700 is implemented on multiple PCA's. In some embodiments, plates 705, 710 may be implemented as parallel lines that follow near seams in the ultrasound probe housing and/or other areas vulnerable to fluid ingress. In some embodiments, the capacitive structure 700 may be implemented on a flexible circuit.

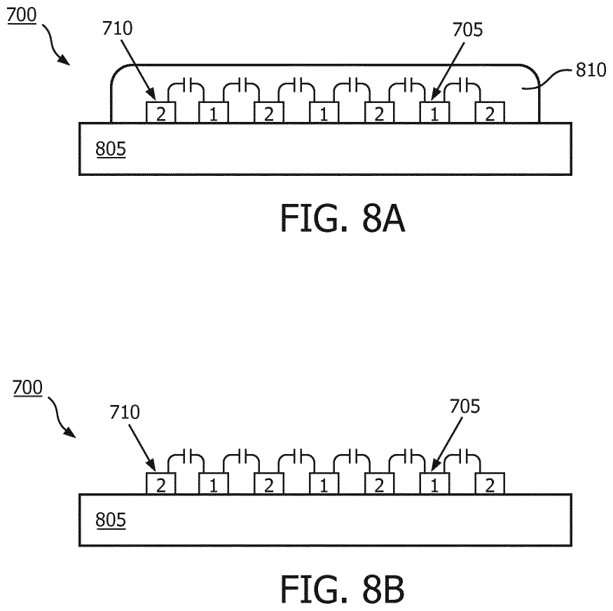

FIGS. 8A and 8B are illustrations of cross-sectional views of capacitive structure 700 according to embodiments of the disclosure. In the example shown in FIGS. 8A and 8B, plates 705, 710 are implemented on a PCA 805. FIG. 8A shows plates 705, 710 covered in a soluble dielectric 810 prior to a fluid ingress. Soluble dielectric 810 coats plates 705, 710 and fills the spaces between the “fingers.” FIG. 8B shows the capacitive structure 700 after a fluid ingress when some or all of soluble dielectric 810 has reacted and/or dissolved. Capacitive structure 700 may have a capacitance of 1-100 pF. The capacitance may be based, at least in part, on the size of the capacitive structure 700, the number of capacitive structures 700 included in an ultrasound probe, a desired sensitivity of a fluid ingress detector, and/or the power specifications of the ultrasound probe. Depending on the nature of a fluid that invades the interior of the ultrasound probe, the fluid may leave a residue on the plates 705, 710 even though some or all of the soluble dielectric 810 is dissolved. In some embodiments, the size of capacitance structure 700, spacing between plates 705, 710, and/or a sensitivity of a dielectric property sensing circuit (e.g., dielectric sensing circuit 620) may be selected based, at least in part, on the effects of the residue.

Capacitive structure 700 shown in FIGS. 7, 8A, and 8B is provided for exemplary purposes. Other capacitive structures may be used to implement capacitors 625, 635 in other embodiments.

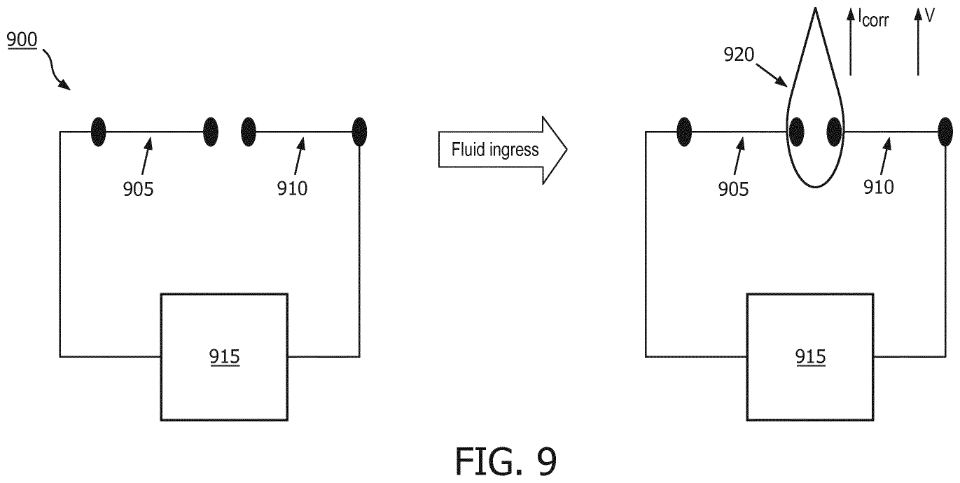

FIG. 9 is an illustration of an example fluid ingress detector 900 according to an embodiment of the disclosure. Fluid ingress detector 900 includes first portion 905 and a second portion 910. In some embodiments, the first portion 905 and second portion 910 may be implemented as conductive wires, traces, and/or plates. In some embodiments, the first portion 905 is implemented as a wire, trace, and/or plate and the second portion 910 is an interior surface of an ultrasound probe housing, for example, interior surface 221 of housing 222 of ultrasound probe 200 shown in FIG. 2. In another example, the second portion 910 is a graphite block of a backing subassembly of an ultrasound probe, for example, backing subassembly 214 shown in FIG. 2. The first portion 905 and second portion 910 may be different materials to form a galvanic sensor. For example, first portion 905 may be copper or a copper alloy and the second portion 910 may be aluminum, nickel-plated aluminum, or an aluminum alloy. In some embodiments, an ultrasound probe may include multiple fluid ingress detectors 900.

As shown on the left-hand side of FIG. 9, prior to a fluid ingress, first portion 905 and second portion 910 are not electrically coupled, forming an open circuit. Other sections of the first portion 905 and second portion 910 may be coupled to a sense circuit 915 in some embodiments. As shown on the right-hand side of FIG. 9, during a fluid ingress, a fluid 920 may, at least temporarily, electrically couple first portion 905 and second portion 910. This may form a closed circuit between the first portion 905 and the second portion 910. In some embodiments, the fluid 920 may include a strong acid, base, and/or oxidative compound that may have a corrosive effect on the first portion 905 and/or second portion 910. The galvanic sensor may generate a current and/or voltage responsive, at least in part, to the fluid 920. The sense circuit 915 electrically coupled to the first portion 905 and second portion 910 may detect a change in voltage, resistance, and/or current. The detected change in voltage, resistance, and/or current may cause the sense circuit 915 to transmit a signal indicating a fluid ingress. The signal may be provided to another component within the ultrasound probe and/or an external system coupled to the ultrasound probe (not shown).

FIGS. 3-9 illustrate examples of fluid ingress detectors according to embodiments of the disclosure. An ultrasound probe and/or ultrasound imaging system may include a fluid ingress detection assembly including one or more of the example fluid ingress detectors. The fluid ingress detectors may be the same or may be different detector types. For example, an ultrasound probe, such as ultrasound probe 12 shown in FIG. 1 or ultrasound probe 200 shown in FIG. 2, may include fluid ingress detector 300 and fluid ingress detector 400. In another example, an ultrasound probe may include fluid ingress detector 500A and an ultrasound imaging system may include fluid ingress detector 600A. The examples are provided for illustrative purposes only, and other configurations of ultrasound probes and/or ultrasound imaging systems including fluid ingress detection assemblies as described herein may be implemented.

In some embodiments, a fluid ingress detection assembly including one or more of the fluid ingress detectors described in reference to FIGS. 3-9 may not include power sources. That is, the fluid ingress detection assembly may only detect and/or provide signals indicating fluid ingress when the ultrasound probe is coupled to an ultrasound imaging system or other external device that provides a power source. For example, a fluid ingress detector may be operable to detect a fluid ingress even without a power source, but the fluid ingress detection assembly may not provide an indication of the detected fluid ingress until the ultrasound probe is coupled to an ultrasound imaging system. In another example, a fluid ingress that occurs while the ultrasound probe is disconnected may not be detected until the ultrasound probe is coupled to an ultrasound imaging system and/or other power source.

In some embodiments, the fluid ingress detection assembly includes a battery or is electrically coupled to a battery included with the ultrasound probe. The fluid ingress detector of the fluid ingress detection assembly may detect fluid ingress and/or provide signals indicating fluid ingress when the ultrasound probe is not coupled to an external power source. For example, a fluid ingress detection assembly may provide a visual indicator (e.g., LED) on the exterior of an ultrasound probe even when the ultrasound probe is not coupled to an ultrasound imaging system. In some embodiments, such as the galvanic sensor-based fluid ingress detector shown in FIG. 9, the fluid may provide a power source for the fluid ingress detector and/or fluid ingress detection assembly.

In some embodiments, the fluid ingress detection assembly may include or may be electrically coupled to a switch, latch, and/or clock. When a fluid ingress occurs, the external, battery, and/or galvanic power may allow a trigger of the switch, latch, and/or clock. The fluid ingress detector of the fluid ingress detection assembly may trigger the switch, latch, and/or clock via a sense circuit included with the fluid ingress detection assembly and/or electrically coupled to the fluid ingress detection assembly. The switch and/or latch may store a state that indicates a fluid ingress occurred. The clock may store a state that indicates a time when the fluid ingress occurred. The states of the switch, latch, and/or clock may be read the next time the ultrasound probe is coupled to a power source and/or a diagnostic process is run on the ultrasound probe by an ultrasound imaging system and/or diagnostic maintenance system.

FIG. 10 shows a circuit diagram of an exemplary sense circuit 1000 according to embodiments of the disclosure. Sense circuit 1000 may be coupled to the fluid ingress detectors described in reference to FIGS. 3-9. Sense circuit 1000 may be coupled to a fluid ingress detector 1005. Fluid ingress detector 1005 may be implemented by one or more of the fluid ingress detectors described in FIGS. 3-9. In some embodiments, sense circuit 1000 may be included as an integral component of the fluid ingress detector 1005. The fluid ingress detector 1005 may be coupled to a first resistance 1010 and a positive input of an operational amplifier (op-amp) 1015. As shown, in some embodiments, the op-amp 1015 may be configured as a differential amplifier. The first resistance 1010 may be further coupled to a common voltage (e.g. 0 V, ground). The negative input of the op-amp 1015 may be coupled to a second resistance 1020. The second resistance 1020 may be further coupled to the common voltage. The negative input of the op-amp 1015 and the output of the op-amp 1015 may be coupled by a third resistance 1025 and a capacitance 1030 coupled in parallel. The output of the op-amp 1015 may further be coupled to a fourth resistance 1035. The fourth resistance 1035 may also be coupled to the base of a transistor 1040. In some embodiments, the transistor 1040 is a NPN bipolar junction transistor. In some embodiments, the fourth resistance 1035 is omitted, and the output of op-amp 1015 is coupled to the base of transistor 1040. The collector of the transistor 1040 may be coupled to a voltage source V₁. The emitter of the transistor 1040 may be coupled to the anode of a first diode 1045. The cathode of first diode 1045 may be coupled to a fuse 1050, which may be further coupled to the common voltage. The cathode of first diode 1045 may be further coupled to a fifth resistance 1065. The fifth resistance 1065 may be coupled to a sixth resistance 1060 and the cathode of a second diode 1055. The anode of the second diode 1055 may be coupled to a voltage source V₂.

A change in the voltage and/or current provided by the fluid ingress detector 1005 to the sense circuit 1000 may be amplified and output by the op-amp 1015. The output may activate the transistor 1040 and allow a current to flow through the diode 1045 and generate an output. The output of the sense circuit 1000 may be provided to an external system 1070. In some embodiments, external system 1070 may be an ultrasound imaging system coupled to an ultrasound probe including sense circuit 1000. In some embodiments, external system 1070 may be a maintenance diagnostic system coupled to an ultrasound probe including sense circuit 1000. In some embodiments, external system 1070 may be internal to an ultrasound probe that includes sense circuit 1000, for example, an ultrasound probe that includes a fluid ingress indicator (e.g., LED, LCD screen, audio signal).

In an example embodiment of sense circuit 1000, resistance 1010 may have a resistance of 1 MΩ, resistance 1020 may have a resistance of 1 kΩ, resistance 1025 may have a resistance of 20 kΩ, resistance 1065 may have a resistance of 1 kΩ, and resistance 1060 may have a resistance of 1 kΩ. Continuing this example, capacitance 1030 may have a capacitance of 0.01 μF. Voltage sources V₁ and V₂ may provide +5 V. Fuse 1050 may be a 50 mA fast fuse. Fuse 1050 may be a 100 mA fast fuse. Diodes 1045, 1055 may be 1N4002 diodes. Transistor 1040 may be a 2N3904 transistor. The values of the resistances, capacitance, fuse, and voltage sources of sense circuit 1000 may be selected based, at least in part, on the power specifications of the ultrasound probe, power specifications of the ultrasound imaging system, the sensitivity of the fluid ingress detector, and/or power specifications of a maintenance diagnostic system.

Sense circuit 1000 is provided merely as an example sense circuit that may be used to implement one or more embodiments of the disclosure. Other sense circuits known in the art may be used to implement a sense circuit coupled to a fluid ingress detector.

Although the present system has been described with reference to an ultrasound imaging system, the present system may be extended to other ultrasound transducers. Additionally, the present system may be used to obtain and/or record image information related to, but not limited to renal, testicular, prostate, breast, ovarian, uterine, thyroid, hepatic, lung, musculoskeletal, splenic, nervous, cardiac, arterial and vascular systems, as well as other imaging applications related to ultrasound-guided interventions and other interventions which may be guided by real-time medical imaging. Further, the present system may also include one or more elements which may be used with non-ultrasound imaging systems with or without real-time imaging components so that they may provide features and advantages of the present system.

Further, the present methods, systems, and apparatuses may be applied to existing imaging systems such as, for example, ultrasonic imaging systems. Suitable ultrasonic imaging systems may include a Philips® ultrasound system which may, for example, support a conventional broadband linear array transducer that may be suitable for small-parts imaging.

Certain additional advantages and features of this invention may be apparent to those skilled in the art upon studying the disclosure, or may be experienced by persons employing the novel system and method of the present invention, chief of which is detection of fluid ingress in ultrasound transducers and method of operation thereof is provided. Another advantage of the present systems and method is that conventional medical imaging systems may be easily upgraded to incorporate the features and advantages of the present systems, devices, and methods.

Of course, it is to be appreciated that any one of the above embodiments or processes may be combined with one or more other embodiments and/or processes or be separated and/or performed amongst separate devices or device portions in accordance with the present systems, devices and methods.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

1. An ultrasound probe comprising:
a transducer assembly including an array of transducer elements;
a housing enclosing at least a portion of the transducer assembly;
a cable coupled to the housing;
a connector coupled to the cable, the connector configured to couple the ultrasound probe to an ultrasound imaging system; and
a fluid ingress detection assembly comprising:
a sense circuit, and
a fluid ingress detector configured to change from a first state to a second state responsive to ingress of fluid in an interior of the ultrasound probe, and wherein the sense circuit is operable to detect the change from the first state to the second state and generate a signal indicative of fluid ingress.

2. The ultrasound probe of claim 1, wherein the fluid ingress detector comprises:
a first conductive line;
a second conductive line, wherein the first and second conductive lines are electrically coupled to the sense circuit;
a conductive cantilever electrically coupled to the first conductive line; and
a soluble epoxy, wherein the soluble epoxy electrically couples the conductive cantilever to the second conductive line.

3. The ultrasound probe of claim 2, wherein the fluid ingress detector further comprises a spring coupled between the first conductive line and the conductive cantilever, wherein the spring biases the conductive cantilever away from the second conductive line.

4. The ultrasound probe of claim 2, wherein the fluid ingress detector further comprises a lever coupled between the first conductive line and the conductive cantilever, wherein the lever biases the conductive cantilever away from the second conductive line.

5. The ultrasound probe of claim 2, wherein the conductive cantilever is molded such that the conductive cantilever is biased away from the second conductive line.

6. The ultrasound probe of claim 2, wherein the sense circuit is configured to sense at least one of a change in resistance, capacitance, and current when at least a portion of the soluble epoxy dissolves.

7. The ultrasound probe of claim 1, wherein the fluid ingress detector comprises:
a first conductive line;
a second conductive line, wherein the first and second conductive lines are electrically coupled to the sense circuit;
a conductive cantilever electrically coupled to the first conductive line; and
a soluble epoxy, wherein the soluble epoxy electrically isolates the conductive cantilever and the second conductive line.

8. The ultrasound probe of claim 7, wherein the fluid ingress detector further comprises a spring coupled between the first conductive line and the conductive cantilever, wherein the spring biases the conductive cantilever toward the second conductive line.

9. The ultrasound probe of claim 7, wherein the fluid ingress detector further comprises a lever coupled between the first conductive line and the conductive cantilever, wherein the lever biases the conductive cantilever toward the second conductive line.

10. The ultrasound probe of claim 7, wherein the conductive cantilever is molded such that the conductive cantilever is biased toward the second conductive line.

11. The ultrasound probe of claim 7, wherein the sense circuit is configured to sense at least one of a change in resistance, capacitance, and current when at least a portion of the soluble epoxy dissolves.

12. The ultrasound probe of claim 1, wherein the fluid ingress detector comprises:
a light source;
a soluble epoxy coating the light source, wherein the soluble epoxy blocks a light of the light source; and
a photodetector electrically coupled to the sense circuit, wherein the photodetector is configured to detect the light from the light source when at least some of the soluble epoxy dissolves and to generate a voltage or a current responsive to detecting the light of the light source, and wherein the sense circuit is configured to do something with the voltage or current.

13. The ultrasound probe of claim 12, wherein the fluid ingress detector includes a plurality of light sources coated by the soluble epoxy.

14. The ultrasound probe of claim 12, wherein the light source includes an infrared light source.

15. The ultrasound probe of claim 12, wherein the photodetector includes a photodiode.

16. The ultrasound probe of claim 1, wherein the fluid ingress detector comprises:
a first portion comprising a first material; and
a second portion comprising a second material different from the first material, wherein the first portion and second portion comprise a galvanic sensor;
wherein the first and second portions form a closed circuit when a fluid contacts the first and second portions and the sense circuit is configured to sense at least one of a voltage and a current from the galvanic sensor when the closed circuit is formed.

17. The ultrasound probe of claim 16, wherein the first material comprises copper.

18. The ultrasound probe of claim 16, wherein the second material comprises aluminum.

19. The ultrasound probe of claim 1, further comprising a latch electrically coupled to the sense circuit, wherein the sense circuit triggers the latch to store a state indicating a fluid ingress when the sense circuit detects the change from the first state to the second state.

20. The ultrasound probe of claim 1, further comprising a clock electrically coupled to the sense circuit, wherein the sense circuit triggers the clock to store a time of a fluid ingress when the sense circuit detects the change from the first state to the second state.

21. An ultrasound imaging system comprising:
an ultrasound probe including a capacitor including a soluble dielectric; and
a dielectric property sensing circuit electrically coupled to the ultrasound probe, wherein the capacitance sensing circuit is configured to detect a change in a dielectric property of the capacitor when at least some of the soluble dielectric reacts.

22. The ultrasound system of claim 21, wherein the dielectric property sensing circuit is included with the ultrasound probe.

23. The ultrasound system of claim 21, wherein the capacitor comprises a first plate and a second plate spaced from the first plate, wherein the soluble dielectric coats the first and second plates and fills a space between the first and second plates.

24. The ultrasound system of claim 23, wherein the first place and second plate form two parallel lines.

25. The ultrasound system of claim 21, wherein the capacitor is implemented on a printed circuit assembly.
